(19) United States
(12) Patent Application Publication
(10) Pub. No.: US 2012/0128583 A1
(43) Pub. Date: May 24, 2012

(54) DTPA DERIVATIVE, METAL COMPLEX, MR AND CT CONTRAST AGENT AND METHOD FOR MANUFACTURING SAME

(75) Inventors: Tae Jung Kim, Gyeongsangbuk-do (KR); Yong Min Jang, Daegu (KR); Ji Ae Park, Daegu (KR)

(73) Assignee: Kyungpook National University Industry Academic Cooperation Foundation, Daegu (KR)

(21) Appl. No.: 13/388,591

(22) PCT Filed: Dec. 13, 2009
(86) PCT No.: PCT/KR2009/007181

§ 371 Date: Feb. 2, 2012

(30) Foreign Application Priority Data
Aug. 6, 2009 (KR) 10-2009-0072523

(51) Int. Cl. A61K 49/04; C07F 5/00; C07F 13/00; A61K 51/04; B05D 5/00; B05D 7/00; C07C 323/58; A61K 49/18; B82Y 5/00; B82Y 40/00

(52) U.S. Cl. 424/1.37; 560/147; 534/16; 556/50; 534/14; 556/1; 424/9.42; 424/9.32; 427/2.11; 977/773; 977/810; 977/892; 977/930; 977/928

(57) ABSTRACT

The present invention relates to DTPA derivatives capable of forming complexes by combining with metals and the like, metal complexes formed by combining with the DTPA derivatives, MR and CT contrast agents including gold (Au) nano-particles of which surfaces are coated with the metal complexes, and a method for manufacturing the same. The MR and CT contrast agents according to the present invention have a high magnetic relaxation rate, thereby providing an excellent contrast enforcement effect and a long image acquisition time. Furthermore, the MR and CT contrast agents are not toxic to the human body, and are image contrast agents of dual molecules capable of being applied to both MR and CT.

### CROSS-REFERENCE TO RELATED APPLICATION

This application claims priority to and the benefit of Korean Patent Application No. 10-2009-0072523, filed on Aug. 6, 2009, the entire content of which is incorporated herein by reference.

### BACKGROUND OF THE INVENTION

1. Field of the Invention

Embodiments of the present invention relate to DTPA derivatives capable of forming complexes by combining with metals and the like, metal complexes formed by combining with the DTPA derivatives, magnetic resonance (MR) and computed tomography (CT) contrast agents including gold (Au) nano-particles of which surfaces are coated with the metal complexes, and a method for manufacturing the same. The MR and CT contrast agents according to the present invention have a high magnetic relaxation rate, thereby providing an excellent contrast enforcement effect and a long image acquisition time. Furthermore, the MR and CT contrast agents are not toxic to the human body, and are image contrast agents of dual molecules capable of being applied to both MR and CT.

2. Description of the Related Art

X-ray computed tomography (CT) developed in the early 1970s and magnetic resonance imaging (MRI) developed in the 1980s are typical advanced imaging equipment in the radiology and have important roles in offering anatomical information of human body.

Magnetic resonance imaging (MRI) is an advanced technology of obtaining detailed internal images of the human body in a non-invasive way by indicating different signal intensities according to the magnetic relaxation rate generated due to a structural difference between biological tissues. According to MRI, however, an image may be poor in view of definition or visibility and accuracy. Therefore, an image contrast may be increased by reducing a magnetic relaxation rate of T1 or T2 in the human body tissue. In addition, in order to increase sensitivity to lesion, a contrast agent may be used. Accordingly, there is an urgent need for development of a contrast agent for MRI to achieve efficient contrast.

While X-ray based Computed Tomography (CT) is effective in imaging a high-density structure like bones, it demonstrates a considerably reduced definition or visibility in imaging soft structures. That is to say, the X-ray CT has a limitation in defining an imaged structure and its peripheral portions. Therefore, in order to overcome this problem, various studies and advances in the CT contrast agent are highly required. Currently, iodine-based contrast agents, such as Ultravist, are widely used for CT. However, since the iodine-based contrast agent has a very small molecular weight, it may be removed through the kidney within a short time, resulting in a short imaging time. In addition, since the iodine-based contrast agent is momentarily collected in the kidney to then be discharged, kidney toxicity may be caused.

### SUMMARY OF THE INVENTION

Embodiments of the present invention provide DTPA derivatives capable of forming complexes by combining with metals and the like, metal complexes formed by combining with the DTPA derivatives, magnetic resonance (MR) and computed tomography (CT) contrast agents including gold (Au) nano-particles of which surfaces are coated with the metal complexes, and a method for manufacturing the same.

According to an embodiment of the present invention, a diethylenetriaminepentaacetic acid (DTPA) derivative is provided, the DTPA derivative capable of forming complexes by combining with metals, which is represented by the following Chemical Formula (1):

Change under Regulation 91 dated Apr. 13, 2010]

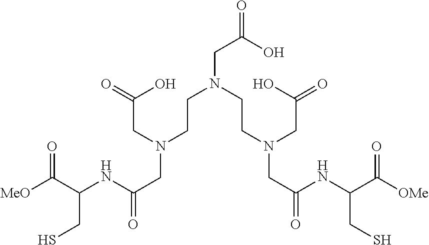

According to another embodiment of the present invention, a metal complex is provided, the metal complex formed by combining a diethylenetriaminepentaacetic acid (DTPA) derivative represented by the Chemical Formula 1 and a metal, which is represented by the following Chemical Formula 2:

[Change under Regulation 91 dated Apr. 13, 2010]

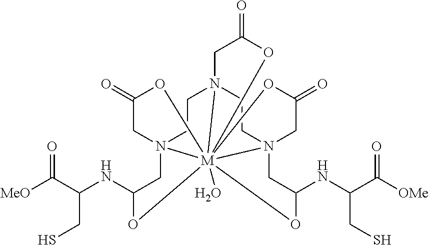

wherein M is selected from the group consisting of Y, Lu, Mn, Tc, Re, Ga, In and lanthanide metals.

Here, M may be gadolinium in the Chemical Formula 2, so that the metal complex may be a gadolinium complex (GdL).

According to still another embodiment of the present invention, MR and CT contrast agents are provided, the MR and CT contrast agents including gold (Au) nano-particles of which surfaces are coated with the metal complex represented by the Chemical Formula 2.

The metal complex may be a gadolinium complex (GdL).

The gold (Au) nano-particles may have a size in a range of 5 to 50 nm.

According to still another embodiment of the present invention, a method for manufacturing MR and CT contrast agents is provided, the method including forming a ligand by reacting a DTPA derivative and an amino acid containing a mercapto group, its derivative or an aromatic compound including a mercapto group and an amino group; forming a metal complex by reacting the ligand with a metal oxide; and coating gold (Au) nano-particles with the complex by dipping the complex in a gold (Au) nano-particle solution.

The DTPA derivative may be a DTPA-bis-anhydride.

The amino acid containing a mercapto group or its derivative may be cysteine, cystine, homocystine, aminothiadiazolethiol, penicilliamine or glutathione.

The amino acid containing a mercapto group may be cysteine.

In the method, M may be gadolinium in the Chemical Formula 2, so that the metal complex may be a gadolinium complex (GdL).

Additional aspects and/or advantages of the invention will be set forth in part in the description which follows and, in part, will be obvious from the description, or may be learned by practice of the invention.

As described above, the contrast agent according to the present invention is a bimodal imaging contrast agent used for both MR and CT.

The contrast agent according to the present invention demonstrates a high magnetic relaxation rate and a contrast enhancement effect, thereby providing more accurate, clear image. Therefore, the contrast agent according to the present invention can offer the same contrast even by use of a smaller amount than in the conventional contrast agent.

The contrast agent according to the present invention is discharged slowly, providing a longer imaging time.

The contrast agent according to the present invention does not cause toxicity into the human body.

Since the contrast agent according to the present invention is specific to the liver, it can be advantageously used for diagnosis of liver cancer.

### BRIEF DESCRIPTION OF THE DRAWINGS

The objects, features and advantages of the present invention will be more apparent from the following detailed description in conjunction with the accompanying drawings, in which:

FIG. 1 briefly illustrates a synthesis process of DTPA derivatives, GdLs and gold (Au) nano-particles coated with GdLs, according to an embodiment of the present invention;

FIG. 2 shows MALDI-TOF mass spectrum results of GdLs according to an embodiment of the present invention;

FIG. 3 shows TEM results of gold (Au) nano-particles of which surfaces are coated with GdLs according to an embodiment of the present invention;

FIG. 4 shows DLS results illustrating a distribution of sizes of gold (Au) nano-particles of which surfaces are coated with GdLs according to an embodiment of the present invention;

FIG. 5 shows FT-IR results of gold (Au) nano-particles of which surfaces are coated with GdLs according to an embodiment of the present invention;

FIG. 6 shows UV spectral result of gold (Au) nano-particles of which surfaces are coated with GdLs according to an embodiment of the present invention;

FIG. 7 is a R₁ graphical representation depending on a change in gadolinium concentrations of a gadolinium complex (GdL) according to an embodiment of the present invention, gadolinium-containing gold (Au) nano-particles (Au@GdL) and omniscan;

FIG. 8 illustrates R₁ maps of a gadolinium complex (GdL) according to an embodiment of the present invention, gadolinium-containing gold (Au) nano-particles (Au@GdL) and omniscan;

FIG. 9 shows MRI T1 enhanced images before and after injecting gold (Au) nano-particles (Au@GdL) to normal mice (ICR 6W, 30 g BW), in which H means heart, L means liver, K means kidney, and B means bladder;

FIG. 10 shows MRI T1 enhanced images before and after injecting GdLs according to an embodiment of the present invention and gold (Au) nano-particles coated with GdLs to normal mice;

FIG. 11 is a graph illustrating liver signal changes over time in MRI T1 enhanced images obtained by injecting GdLs according to an embodiment of the present invention and gold (Au) nano-particles coated with GdLs to normal mice;

FIG. 12 shows CT coefficients of gold (Au) nano-particles (Au@GdL) coated with GdLs according to an embodiment of the present invention, gold (Au) nano-particles (Au@L) not coated with GdLs, and ultravist;

FIG. 13 shows micro CT images before and after injecting gold (Au) nano-particles (Au@GdL) to normal mice (ICR 6W, 30 g BW);

FIG. 14 is a graph illustrating signal changes of various organs over time in micro CT images obtained by injecting gold (Au) nano-particles (Au@GdL) coated with GdL according to an embodiment of the present invention to normal mice;

FIG. 15 is a graph illustrating liver signal changes over time in micro-CT images obtained by injecting to normal mice gold (Au) nano-particles (Au@GdL) coated with GdL according to an embodiment of the present invention and gold (Au) nano-particles (Au@GdL) not coated with GdL;

FIG. 16 shows microscope images of liver tissues sampled 5 hours after injecting gold (Au) nano-particles (Au@GdL) to normal mice, in which (A) is a photograph of normal liver tissues, and (B) is a photograph of liver tissues with Au@GdL injected thereto; and

FIG. 17 shows MIT test results of cell toxicity of gold (Au) nano-particles (Au@GdL) coated with GdL.

### DETAILED DESCRIPTION OF THE INVENTION

Hereinafter, embodiments of the present invention will be described in detail with reference to the accompanying drawings such that they can easily be made and used by those skilled in the art.

The present invention provides diethylenetriaminepentaacetic acid (DTPA) derivatives.

The DTPA derivative according to the present invention has an excellent binding capability through a coordinate covalent bond with a metal to be capable of forming a metal complex, which may be used for a contrast agent. In addition, since the DTPA has a function of discharging radioactive materials from the body, a contrast agent using DTPA or its derivatives has weak toxicity to the human body.

The DTPA derivative may be represented by the following Chemical Formula 1:

[Change under Regulation 91 dated Apr. 13, 2010]

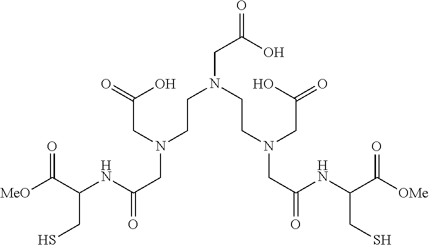

The metal complex formed from the DTPA derivative represented by the Chemical Formula 1 coordinate-covalently bonded with metal as a ligand can be represented by the following Chemical Formula 2:

[Change under Regulation 91 dated Apr. 13, 2010]

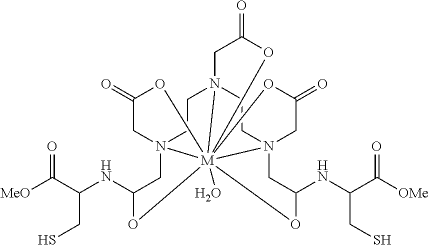

In Chemical Formula 2, the metal complex may be used in preparing an MR or CT contrast agent. In the present invention, a complex is made of a group of atoms formed by coordinating some other atom ionic molecules or group of atoms in 3D with directivity around one or more atoms or ions. Here, the atom ionic molecules or group of atoms coordinated around the central atom or ion are called a ligand. In Chemical Formula 2, metal atom is a central ion and the DTPA derivative is a ligand.

In Chemical Formula 2, M is a metal bonded to the DTPA derivative represented by the Chemical Formula 1. The metal may be a lanthanide such as yttrium (Y), ruthenium (Lu), manganese (Mn), technetium (Tc), rhenium (Re), gallium (Ga), or indium (In). Specific examples of the lanthanide may include gadolinium (Ga), holmium (Ho), dysprosium (Dy), samarium (Sm), and lanthanum (La), preferably gadolinium (Ga).

The metal complex formed by binding the DTPA derivative represented by the Chemical Formula 1 with gadolinium can be represented by Chemical Formula 3:

[Change under Regulation 91 dated Apr. 13, 2010]

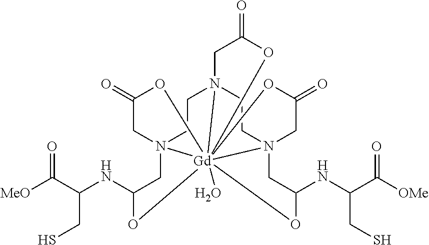

The present invention provides a bimodal imaging contrast agent compatibly used for MR and CT. The MR and CT contrast agent according to the present invention includes gold (Au) nano-particles coated with the metal complex represented by the Chemical Formula 2.

The MR and CT contrast agent including gold (Au) nano-particles coated with the metal complex represented by the Chemical Formula 2 is a bimodal imaging contrast agent that can be used for both MR and CT and has an excellent contrast enhancing effect and a prolonged imaging time.

In the metal complex represented by the Chemical Formula 2, the metal may be gadolinium. Since the gold (Au) and gadolinium (Gd) have higher densities than iodine (I) that is commonly used in a contrast agent for CT (Au: 19.3, Gd: 7.9, I: 4.94), and Gd-containing Au nano-particles have a large molecular weight, the Gd-containing Au nano-particles are discharged slowly in the body, thereby providing an extended imaging time. In addition, since the gold (Au) and gadolinium (Gd) have higher absorption coefficients to X-rays than iodine (I) (Au: 5.158 cm²g⁻¹, Gd: 3.109 cm²g⁻¹, I: 1.94 cm²g⁻¹ at 100 Kev), an increased contrast enhancing effect can be demonstrated.

The gold (Au) nano-particles have a diameter in a range of 5 to 50 nm, preferably in a range of 10 to 20 nm. If the diameter of the gold (Au) nano-particle is less than 5 nm, the gold (Au) nano-particles are discharged from the body within a short time through the kidney. Thus, a time for obtaining an image may be insufficient. If the diameter of the gold (Au) nano-particle is greater than 50 nm, damages may be caused to other organs including the liver.

The MR and CT contrast agent according to the present invention is specific to the liver. In the present invention, the terms ‘being specific’ mean that a relatively large amount of the MR and CT contrast agent according to the present invention accumulates in a specific organ or tissue in the body. Since the MR and CT contrast agent accumulates specifically to the liver, it can be advantageously used in diagnosis and treatment of liver-related diseases.

The present invention also provides a method for manufacturing MR and CT contrast agents, the method comprising: forming a ligand; forming a complex; and coating. According to the above-stated method, gold (Au) nano-particles coated with a metal complex can be prepared. The gold (Au) nano-particles prepared by the above-stated method have reduced structural tumbling mobility of molecules and have a high magnetic relaxation rate, thereby providing an increased contrast enhancing effect. In addition, the gold (Au) nano-particles prepared by the above-stated method have a higher magnetic relaxation rate than other commonly used contrast agents of which each of surfaces is coated with approximately 3000 metal complexes.

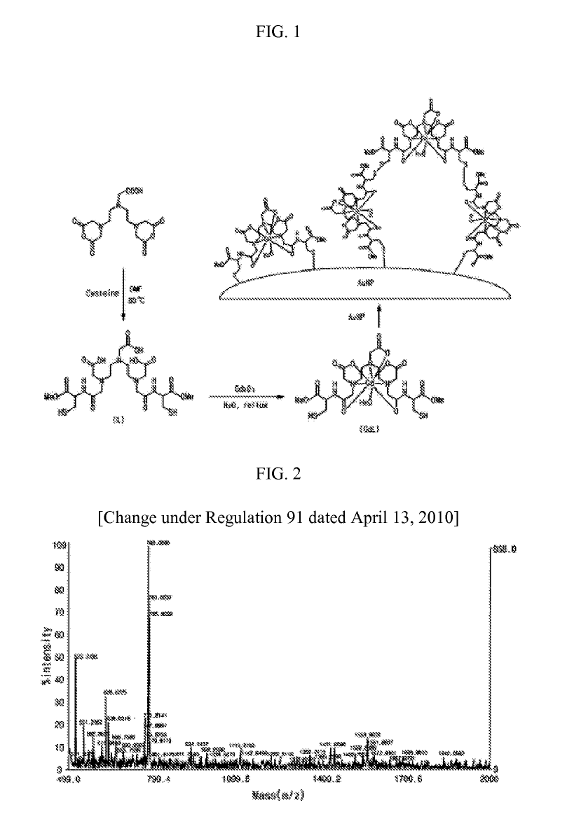

FIG. 1 briefly illustrates a synthesis process of DTPA derivatives, GdL and gold (Au) nano-particles coated with GdL, according to an embodiment of the present invention.

Referring to FIG. 1, in the ligand forming step, the ligand is formed by reacting a DTPA derivative and an amino acid containing a mercapto group, its derivative or an aromatic compound including a mercapto group and an amino group.

The ligand formed in the ligand forming step may be represented by the Chemical Formula 1. The ligand may be obtained by adding the DTPA derivative, the amino acid containing a mercapto group, its derivative or the aromatic compounding including a mercapto group and an amino group in an organic solvent at 50 to 100° C. for 5 to 10 hours.

The DTPA derivative may include non-limiting derivatives that are well known in the art, preferably DTPA-bis-anhydride represented by the following Chemical Formula 4:

[Change under Regulation 91 dated Apr. 13, 2010]

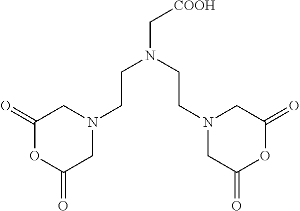

The amino acid containing a mercapto group or its derivative may include commonly used materials, and specific examples thereof may include cysteine, cystine, homocystine, aminothiadiazolethiol, penicilliamine or glutathione, preferably cysteine.

Specific examples of the aromatic compounding including a mercapto group and an amino group may include 4-amino benzenethiol, but not limited thereto.

The organic solvent may include non-limiting organic solvents commonly used in the art, preferably dimethylformamide (DMF). According to an embodiment of the present invention, a cysteine-conjugated DTPA-bis-amide-cysteine ligand can be prepared by reacting DTPA-bis-anhydride represented by the Chemical Formula 4 with cysteine in DMF in an equivalent ratio of 1:2.

In the complex forming step, a metal complex is formed by reacting the ligand formed in the ligand forming step with a metal oxide. In detail, the metal complex may be formed by reacting the ligand formed in the ligand forming step with the metal oxide represented by the Chemical Formula 2.

The metal oxide may include oxides of yttrium (Y), ruthenium (Lu), manganese (Mn), technetium (Tc), rhenium (Re), gallium (Ga), indium (In) or lanthanide, but not limited thereto.

According to an embodiment of the present invention, a gadolinium complex (GdL) represented by the Chemical Formula 3 may be prepared by reacting the DTPA-bis-amide-cysteine ligand formed in the ligand forming step with Gd₃O₃ in water.

In the coating step, gold (Au) nano-particles are coated with the complex by reacting the complex in a gold (Au) nano-particle solution. In the coating step, approximately 3000 or more metal complexes may be bonded to a surface of each of the gold (Au) nano-particles, thereby providing a higher magnetic relaxation rate than other commonly used contrast agents.

The gold (Au) nano-particles may be prepared by the method widely known in the art. Preferably, the gold (Au) nano-particles may be prepared by reducing HAuCl₄ using sodium citrate as a reducing agent. The sizes of the gold (Au) nano-particles may be adjusted by adjusting a ratio of HAuCl₄ to sodium citrate. Preferably, the sizes of the gold (Au) nano-particles may be adjusted in a molar ratio of 1:3 to 1:5 of HAuCl₄ to sodium citrate. In addition, the method for manufacturing the gold (Au) nano-particles may further include heating at a temperature in a range of 130 to 150° C. for 5 to 15 minutes.

According to an embodiment of the present invention, the gold (Au) nano-particles coated with the complex can be prepared by reacting a gadolinium complex (GdL) represented by the Chemical Formula 3 in a solution containing gold (Au) nano-particles well dispersed.

The invention is further illustrated by the following examples and comparative examples, which are not intended to be limiting.

### EXAMPLE 1

### Preparation of DTPA-bis-amide-cystine ligand (L)

DTPA-bis-anhydride (1.13 g, 1 mmol) was added to 15 ml of N,N-dimethylformamide, and stirred to then be added to cystine (L-cysteine methyl ester) (1.09 g, 2 mmol). After the reaction product was stirred at 80° C. for 6 hours, a solvent was completely removed at a low pressure and 5 in of methanol was added thereto to then be dissolved. Then, silica gel (60 mesh) chromatography was performed on the solution using methanol as an eluent, and the solvent was completely removed, followed by drying. A solid matter produced by drying was dissolved in a small amount of methanol and then dropped to a mixed solution containing acetone and ether mixed in a volumetric ratio of 3:7 for precipitation, followed by filtering using a filter, obtaining a white solid. The obtained white solid was dried for 8 hours while a vacuum was maintained at 70° C., thereby obtaining a ligand. The thus obtained ligand is a DTPA derivative represented by the Chemical Formula 1, which is denoted by L in FIG. 1.

### EXAMPLE 2

### Preparation of Gadolinium Complex (GdL)

0.62 g (1 mmol) of the ligand L obtained in Example 1 was added to 10 ml of tertiary distilled water and Gd₂O₃ (0.18 g, 0.5 mmol) was then added thereto. The mixture in a suspension was stirred at 100° C. for 6 hours. Undissolved impurities were completely removed from the suspension by passing the suspension through Celite and the solvent was completely removed. The materials remaining in the suspension were sufficiently dissolved in 5 ml of methanol and reprecipitated in 100 ml of cold acetone. The precipitated white solid was filtered and dried, obtaining the GdL represented by the Chemical Formula 3.

### EXAMPLE 3

### Preparation of Gold (Au) Nano-Particles

HAuCl₄ 3H₂O and water were placed in a three-necked round bottom flask with a condenser and heated with stirring. To the solution was rapidly added sodium citrate (1.14 g, 3.88 mmol), thereby allowing the solution to turn purple from yellow. Heating was maintained at a temperature ranging from 140 to 150° C. for 10 minutes and a heating mantle was then removed, followed by additionally stirring for 10 minutes, thereby obtaining gold (Au) nano-particles. The obtained gold (Au) nano-particles had a size of approximately 15 nm in a well water-dispersed state.

### EXAMPLE 4

### Preparation of Gadolinium Complex (GdL) Coated with Gold (Au) Nano-Particles (Au@GdL)

1.5 g of the gadolinium complex (GdL) prepared in Example 2 was added to a dispersion having 1 l of the gold (Au) nano-particles prepared in Example 3 and reacted in a dark place for 24 hours. After reacting for 24 hours, 1 l of acetone was added to the reaction product to then be reacted for 3 hours, thereby precipitating gold (Au) nano-particles coated with GdL. The precipitated particles were separated by centrifugation (3,600 rpm, 10 minutes), followed by drying, thereby obtaining gold (Au) nano-particles coated with the GdL.

### EXPERIMENTAL EXAMPLE 1

### Identification of DTPA Derivative and Gadolinium Complex (GdL)

ESI-MS was performed on the DTPA derivative prepared in Example 1 and the GdL prepared in Example 2 and the results are as follows.

FIG. 2 shows MALDI-TOF mass spectrum results of GdL according to an embodiment of the present invention.

1. C₂₂H₃₉N₅O₁₃S₂ (L)

Calculated values: C: 40.92, H: 6.09, N: 10.85

Experimental values: C: 40.73, H: 6.34, N: 11.08

Maldi-TOF (m/z): 628.09 ([M+H])

### 2. C₂₂H₃₈GdN₅O₁₄S ([GdL]+2H₂O)

Calculated values: C: 32.30, H: 4.68, N: 8.56

Experimental values: C: 32.47, H: 5.08, N: 8.81

Maldi-TOF (m/z): 783.09 ([M-H₂O+H])

From the results of Experimental Example 1, it was confirmed that the resultant products were identified as the DTPA derivative (L) and the GdL prepared in Examples 1 and 2.

### EXPERIMENTAL EXAMPLE 2

### Measurement of Sizes of Gold (Au) Nano-Particles Coated with Gadolinium Complex (GdL)

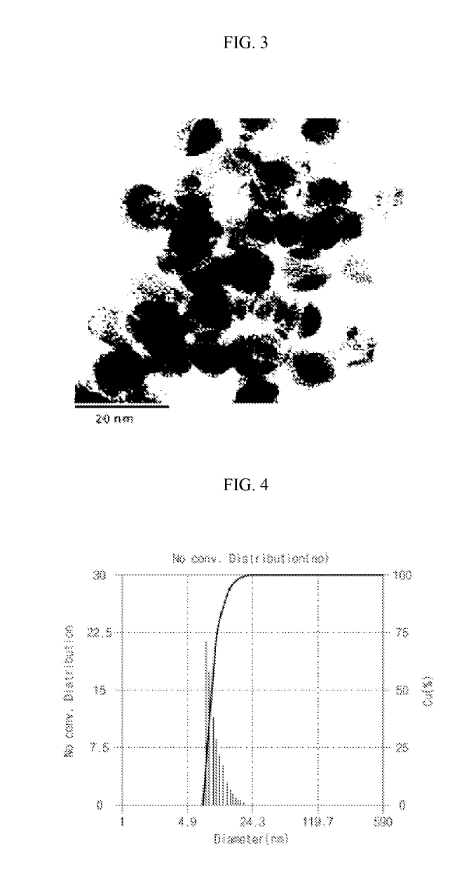

A drop of a solution of gold (Au) nano-particles coated with the GdL prepared in Example 4 was placed on a 200 mesh copper carbon grid and dried at room temperature for transmission electron microscope (TEM) (Philips CM 200) measurement. The TEM measurement was carried out at 200 kV. The TEM measurement result is shown in FIG. 3. In addition, the sizes of gold (Au) nano-particles coated with GdL were measured using dynamic light scattering (DLS), and the results thereof are shown in FIG. 4.

Referring to FIG. 3, the size of the gold (Au) nano-particle coated with GdL was approximately 15 nm. In addition, referring to FIG. 4, average particle sizes, as measured by dynamic light scattering particle size analysis (DLS), were predominantly distributed around 15 nm.

### EXPERIMENTAL EXAMPLE 3

### FT-IR Analysis of Gold (Au) Nano-Particles Coated with GdL

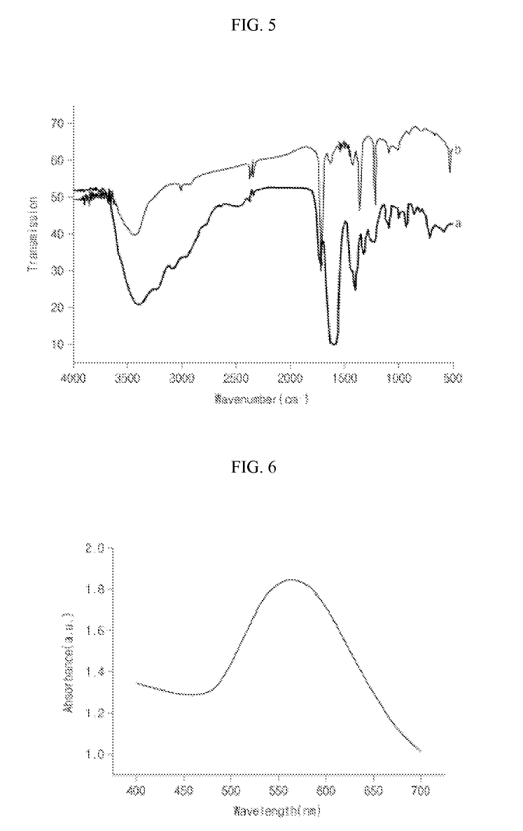

Fourier transform infrared spectroscopy (FT-IR) was performed on the gold (Au) nano-particles coated with GdL prepared in Examples 2 and 4. FT-IR was conducted using a Mattson FT-IR Galaxy 6030E spectrophotometer. The result of FT-IR analysis was shown in FIG. 5. In FIG. 5, the curve ‘a’ indicates IR spectral result for GdL and the curve ‘b’ indicates IR spectral result for Au@GdL.

Referring to FIG. 5, a 2550 cm⁻¹ S-H stretching band appearing on GdL was not demonstrated on Au@GdL, which is because a thiol group in GdL disappeared while forming disulfide bonds between GdL and gold (Au) nano-particles. In addition, a distinct change was also caused to Au@GdL in that the peak intensity was lowered at 1500˜2000 cm⁻¹, which is presumably due to a carboxy group in GdL bonded to a surface of gold (Au).

### EXPERIMENTAL EXAMPLE 4

### UV Spectral Analysis of Gold (Au) Nano-Particles Coated with Gadolinium Complex (GdL)

The UV spectral analysis was performed on the gold (Au) nano-particles (Au@GdL) coated with the GdL prepared in Example 4, and the result thereof is shown in FIG. 6.

Referring to FIG. 6, Au@GdL demonstrated an absorption peak at approximately 540 nm, which involved excitation of surface plasmon vibration.

### EXPERIMENTAL EXAMPLE 5

### Measurement of Number of GdL Coating each Gold (Au) Nano-Particle

The number of GdLs coating each gold (Au) nano-particle was calculated using the particle sizes confirmed by TEM performed on the gold (Au) nano-particles (Au@GdL) coated with GdL prepared in Example 4 (see chemcomm, 2006, 1433-1435), and the result thereof is shown in Table 1 below.

TABLE 1

| No. | Au (ppm) | Gd (ppm) | No. of Gd/AuNP |
|---|---|---|---|
| 1 | 81800 | 1229 | 3100 |
| 2 | 357600 | 5510 | 3170 |
| 3 | 422800 | 62144 | 3050 |
| 4 | 391200 | 5864 | 3062 |

As shown in Table 1, 3000 or more GdLs were coated on each gold (Au) nano-particle. Thus, since the number of GdLs coated was large, a high magnetic relaxation rate and a contrast enhancing effect were demonstrated. Accordingly, it is expected to obtain a more accurate, clear image.

### EXPERIMENTAL EXAMPLE 6

### Measurement of Magnetic Relaxation Rate

Magnetic relaxation rates R₁ and R₂ of the GdL prepared in Example 2 and the gold (Au) nano-particles (Au@GdL) containing the GdL prepared in Example 4 were measured. Relaxation time (T1) measurements were carried out using an inversion recovery method with a variable inversion time (T1) at 1.5 T (64 MHz). A magnetic resonance (MR) image requires 35 different T1 values from a region of 50 to 1750 msec. T₁ was obtained from non-linear, square seizures of signal intensities measured for the respective T1 values. Carr-Purcell-Meiboon-Gill (CPMG) pulse sequences for T₂ measurement are obtained by multiple spin-echo measurements. 34 images may have 34 different values of echo time in a range of 10 to 1900 msec. A relaxation time (T₂) was obtained from non-linear, square pixels for multiple spin-echo measurements at each TE. Relaxivities (R1 and R2) were calculated as an inverse of relaxation time per mM, and the result thereof is shown in Table 2, in which GdL denotes a gadolinium complex prepared in Example 2, and Au@GdL denotes gold (Au) nano-particles coated with GdL prepared in Example 4.

TABLE 2

| Au(ppm) | R1/mM, sec | R2/mM, sec |
|---|---|---|
| Omniscan ® | 3.3 ± 0.03 | 3.8 ± 0.06 |
| GdL | 7.5 ± 0.08 | 12.3 ± 0.21 |
| Au@GdL: [Gd] | 17.9 ± 1.1 | 28.2 ± 1.0 |
| Au@GdL: [AuNP] | 4.6 × 10⁵ | 7.2 × 10⁵ |

The most common and effective way of proving efficiency of a contrast agent is to measures a magnetic relaxation rate. As shown in Tables 2 and 7, the gadolinium complex (GdL) or the gold (Au) nano-particles (Au@GdL) coated with GdL has a magnetic relaxation rate 2 to 3 times higher than that of Omniscan, which is a representative one among commercialized MR contrast agents. Therefore, the contrast agent according to the present invention may be an MRI contrast agent which can effectively indicate a signal.

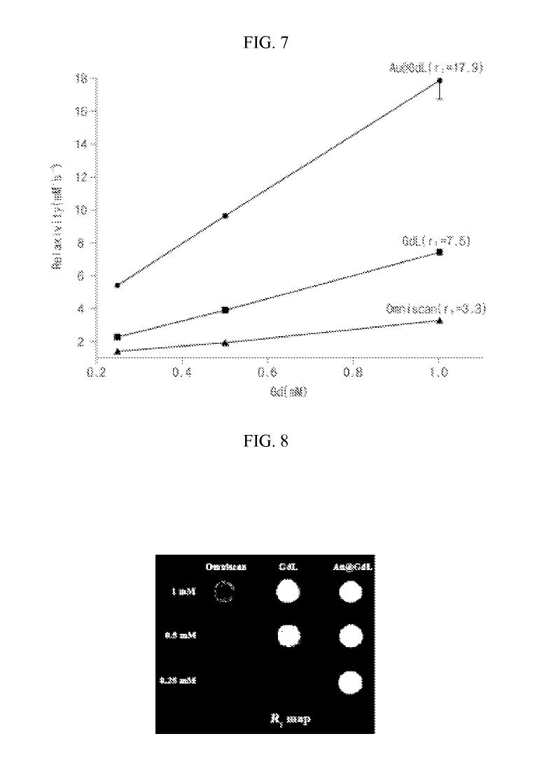

Referring to FIG. 7, signals of the gadolinium complex (GdL) prepared in Example 2 and the gold (Au) nano-particles coated with GdL prepared in Example 4 were increased compared to Omniscan in the same concentration, higher intensity signals were observed from the gold (Au) nano-particles (Au@GdL) coated with GdL than in GdL.

FIG. 8 illustrates R₁ maps of a gadolinium complex (GdL) according to an embodiment of the present invention, gadolinium-containing gold (Au) nano-particles (Au@GdL) coated with GdL and omniscan.

Referring to FIG. 8, a brighter R₁ map was obtained from the gadolinium-containing gold (Au) nano-particles (Au@GdL) coated with GdL prepared in Example 4 than from Omniscan.

### EXPERIMENTAL EXAMPLE 7

### T1 Enhanced Image and Liver Signal Measurement

In order to investigate liver specificity of the gadolinium complex(GdL) prepared in

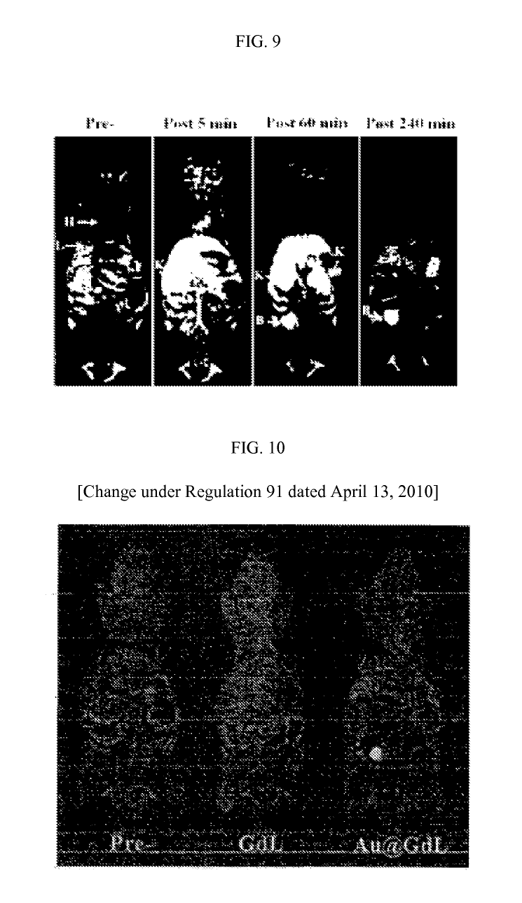

Example 2 and the gadolinium-containing gold (Au) nano-particles (Au@GdL) including GdL prepared in Example 4, an MR enhancement effect and liver signals were measured, and the results thereof are shown in FIGS. 9 to 11. MR images of anaesthetized ICR mice were obtained pre- and post- Au@GdL (0.03 mmol[Gd]/Kg) injection by tail vein with a 1.5 Tesla (T) MR unit (GE Signa Advantage, GE Medical system, USA), and the result thereof is shown in FIG. 9.

FIG. 9 shows MRI T1 enhanced images before and after injecting Au@GdL to normal mice (ICR 6W, 30 g BW) over time. As shown in FIG. 9, there was strong signal enhancement in the mouse liver. In addition, a blood pool effect for abdominal aorta contrast was also demonstrated.

FIG. 10 shows MRI T1 enhanced images of GdL and Au@GdL. As shown in FIG. 10, the brightest image was obtained from the gold (Au) nano-particles including GdL prepared in Example 4.

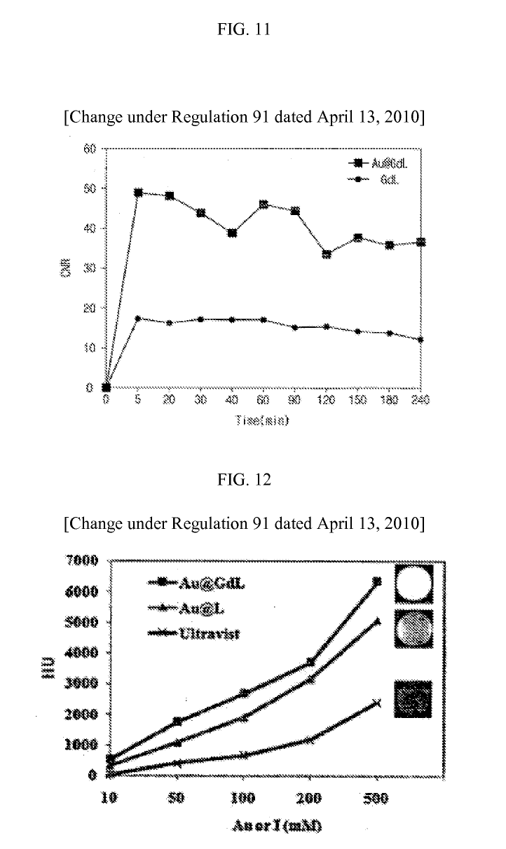

FIG. 11 is a graph illustrating liver signal changes over time in MRI T1 enhanced images obtained by injecting GdL according to an embodiment of the present invention and gold (Au) nano-particles (Au@GdL) coated with GdL to normal mice.

Referring to FIG. 11, signal intensity of Au@GdL was more than 2 times higher than that of GdL.

### EXPERIMENTAL EXAMPLE 8

### CT Signal Measurement

Hounsfield unit (HU) scales of gold (Au) nano-particles (Au@GdL) coated with the GdL prepared in Example 4 and gold (Au) nano-particles (Au@L) not coated with GdL were measured. The HU is a unit indicating a reduction extent of X-rays, that is, relative density of a tissue. That is to say, the larger the HU scale, the higher the absorption coefficient to X-rays, looked bright white in CT scanning. The HU measurement result is shown in FIG. 12. The Ultravist is a currently commercialized iodine-based CT contrast agent.

As shown in FIG. 12, since the gold (Au) nano-ₚarticles (Au@GdL) coated with the GdL prepared in Example 4 and the gold (Au) nano-particles (Au@L) not coated with GdL have higher HU scales than Ultravist, suggesting that the CT contrast agent including the gold (Au) nano-particles (Au@GdL) coated with GdL is capable of more effectively indicating signals. In addition, the gold (Au) nano-particles (Au@GdL) coated with GdL shows a higher signal intensity than the gold (Au) nano-particles (Au@L) not coated with GdL, suggesting that gadolinium (Gd) was contributable to signal enhancement.

### EXPERIMENTAL EXAMPLE 9

### Measurement of CT Contrast Effects

In order to investigate CT contrast effects of the gold (Au) nano-particles (Au@GdL) coated with the GdL prepared in Example 4 and the gold (Au) nano-particles (Au@L) not coated with GdL, CT images were obtained from mice. The CT images were photographed using an INVEON (Siemens Medical Solutions) CT scanner under conditions of 60 kVp, 500 mA; 200-millisecond per frame, a reconstruction image of 512 pixels.

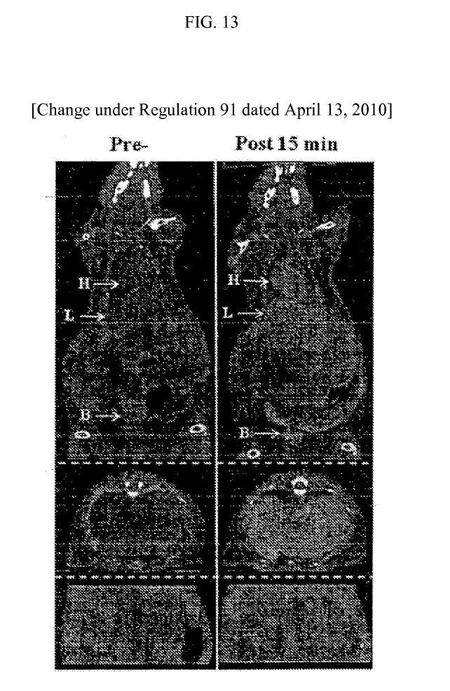

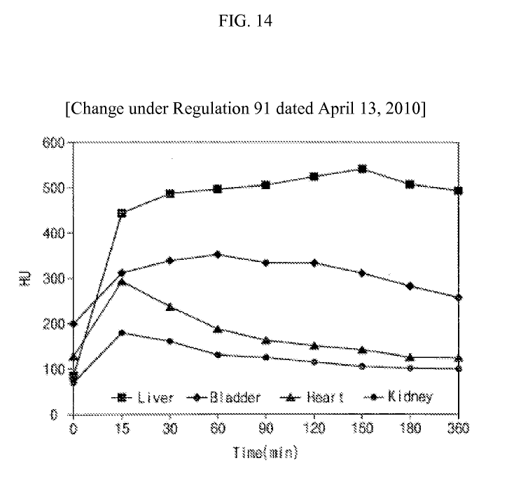

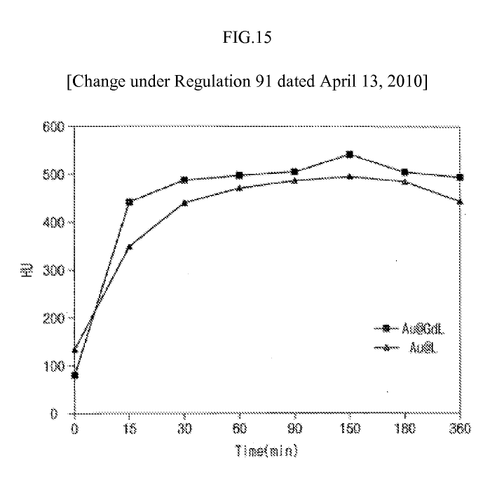

CT images were taken before and after injecting each 1.75 mmol [Au]/kg of Au@GdL and Au@L into mice, and the results thereof are shown in FIGS. 13 to 15. FIG. 13 shows images taken before and 15 minutes after injecting Au@GdL, FIG. 14 shows signals of various organs over time after injecting Au@GdL, and FIG. 15 shows measurement results of liver signals over time from CT images obtained by injecting Au@GdL and Au@L to mice.

Referring to FIG. 13, a higher-intensity signal (a brighter image) was observed from the liver after injecting Au@Gd than before injecting Au@GdL.

Referring to FIG. 15, the liver signal of a mouse into which Au@GdL was injected was intenser than that of a mouse into which Au@L was injected. Therefore, it can be understood that Au@GdL demonstrated excellent contrast enhancing effect.

### EXPERIMENTAL EXAMPLE 10

### Liver Tissue Examination

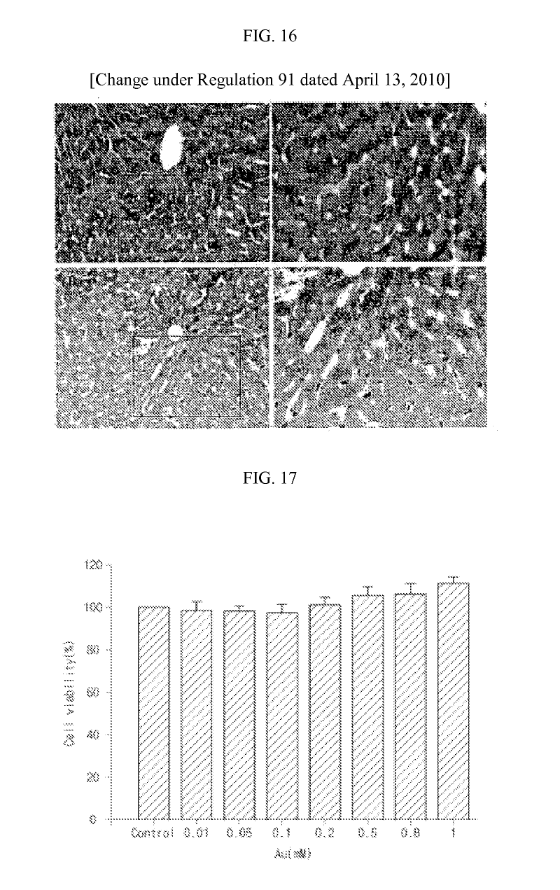

In order to detect an uptake route in MR and CT tests, which was enabled because the liver signal of the mouse into which Au@GdL was injected was intense and was continuously indicated, liver tissue examination was conducted. 5 hours after injecting Au@GdL (0.03 mmol[Gd]/Kg) into tail veins of three mice, cardiovascular perfusion was performed using 4% paraform-aldehyde (pH 7.2) in phosphate buffered saline (PBS). After 5 minute pre-rinsing was performed with 20 ml of PBS, a syringe needle was inserted into the left ventricle to perfuse the left ventricle with 30 ml of PBS for 10 minutes. The removed liver was fixed at 4° C. for one day. The liver was dehydrated, treated with xylene, embedded in paraffin, cut into 3 ml and dyed with hematoxylin and eosin. FIG. 16 shows normal liver tissues taken using a microscope Carl zaiss Axio Image.A1. An AxioCam MRCS camera with 2584×936 resolution was used. In FIG. 16, (A) shows normal mice and (B) shows a liver tissue of a mouse into which Au@GdL was injected. The left photographs are 200 times magnified photographs and the right photographs are 400 times magnified photographs.

Referring to FIG. 16, in the right photograph of (A), a triangle indicates hepatocyte (liver cell) and a long arrow indicates kupffer cells. In (B), black parts indicate nano-particles, suggesting that Au@GdL was selectively taken up only through kupffer cells.

### EXPERIMENTAL EXAMPLE 11

### Measurement Cell Toxicity

In order to investigate cell toxicity, a tetrazolium (MTT) assay was used. The MTT assay uses capability of mitochondria to reduce water-soluble yellow MIT tetrazolium to water-insoluble purple MTT formazan (3-(4,5-dimethylthiazol-2-yl)-2,5-diphenyl-tetrazolium bromide) by the action of a dehydrogenase. If viable (living) cells are treated with MTT tetrazolium, MTT tetrazolium is reduced by a mitochondrial reductase to form MTT formazan (3-(4,5-dimethylthiazol-2-yl)-2,5-diphenyl-tetrazolium bromide). That is to say, if a compound is treated with varying concentrations for a constant time to sufficiently induce apoptosis (cell death) and then treated with MTT tetrazolium, MTT formazan is formed in a low concentration without cell toxicity, while MTT formazan is not formed in a high concentration with cell toxicity. Therefore, cell viability can be determined by measuring the formation extent of MTT formazan depending on the concentration of the compound. 14D Chick cornea stroma primary cells were used as a control group. Au@GdL prepared in Example 4 was added to the same cells with various concentrations in a range of 0.01 to 1 mM Au to measure formation extents of MTT formazan, which were compared with those of MTT formazan of the control group. The cell viability was compared based on 100% of cell viability and the result thereof is shown FIG. 17.

Referring to FIG. 17, when Au@GdL prepared in Example 4 was added in various concentrations, greater than 95% of cell viability was observed in all concentrations, confirming that the cell toxicity of Au@GdL prepared in Example 4 was so low that Au@GdL could be advantageously used as a contrast agent.

As described above, the DTPA derivatives according to the present invention are capable of forming metal complexes by combining with metals etc., the metal complexes are coated on surfaces of gold (Au) nano-particles to be used as MR and CT contrast agents. The gadolinium (Gd)-coated gold (Au) nano-particles (Au@GdL) can be widely used as a bimodal imaging contrast agent compatibly used for MR and CT while having excellent contrast enhancing effect and a prolonged imaging time. In addition, the DTPA derivatives according to the present invention are expected not to induce toxicity to the human body due to low cell toxicity.

While this invention has been described in connection with what is presently considered to be practical exemplary embodiments, it is to be understood that the invention is not limited to the disclosed embodiments, but, rather is intended to cover various modifications and equivalent arrangements included within the spirit and scope of the appended claims.

What is claimed is:

1. A diethylenetriaminepentaacetic acid (DTPA) derivative capable of forming complexes by combining with metals, which is represented by the following Chemical Formula (1):
[Change under Regulation 91 dated Apr. 13, 2010]

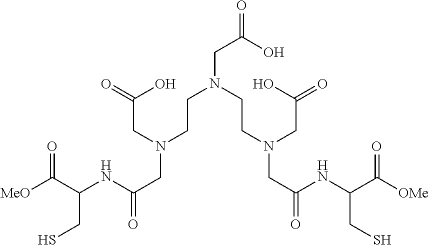

2. A metal complex formed by combining a diethylenetriaminepentaacetic acid (DTPA) derivative represented by the Chemical Formula 1 and a metal, which is represented by the following Chemical Formula 2:
[Change under Regulation 91 dated Apr. 13, 2010] 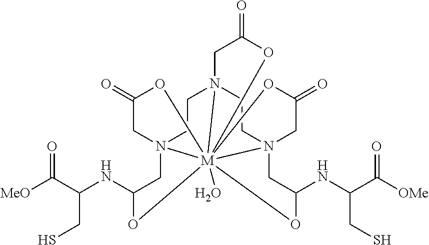
wherein M is selected from the group consisting of Y, Lu, Mn, Tc, Re, Ga, In and lanthanide metals.

3. The metal complex of claim 2, wherein M is gadolinium in the Chemical Formula 2, so that the metal complex is a gadolinium complex (GdL).

4. MR and CT contrast agents comprising gold (Au) nano-particles of which surfaces are coated with the metal complex represented by the Chemical Formula 2.

5. The MR and CT contrast agents of claim 4, wherein the metal complex is a gadolinium complex (GdL).

6. The MR and CT contrast agents of claim 4, wherein the gold (Au) nano-particles have a size in a range of 5 to 50 nm.

7. A method for manufacturing MR and CT contrast agents, the method comprising:
forming a ligand by reacting a DTPA derivative and an amino acid containing a mercapto group, its derivative or an aromatic compound including a mercapto group and an amino group;
forming a metal complex by reacting the ligand with a metal oxide; and
coating gold (Au) nano-particles with the complex by dipping the complex in a gold (Au) nano-particle solution.

8. The method of claim 7, wherein the DTPA derivative is a DTPA-bis-anhydride.

9. The method of claim 7, wherein the amino acid containing a mercapto group or its derivative is cysteine, cystine, homocystine, aminothiadiazolethiol, penicilliamine or glutathione.

10. The method of claim 7, wherein the amino acid containing a mercapto group is cysteine.

11. The method of claim 7, wherein M is gadolinium in the Chemical Formula 2, so that the metal complex is a gadolinium complex (GdL).
